(19) United States
(12) Patent Application Publication
(10) Pub. No.: US 2008/0096280 A1
(43) Pub. Date: Apr. 24, 2008

(54) Method of Assaying the Antioxidant Activity of Oils

(76) Inventors: Somasuntharam Tharmalingam Senthilmohan, Christchurch (NZ); Murray James McEwan, Christchurch (NZ)

Correspondence Address: D. PETER HOCHBERG CO. L.P.A., 1940 EAST 6TH STREET, CLEVELAND, OH, 44114, US

(21) Appl. No.: 11/661,077

(22) PCT Filed: Aug. 22, 2005
(86) PCT No.: PCT/NZ05/00221

§ 371 Date: Feb. 27, 2007

(30) Foreign Application Priority Data
Aug. 26, 2004 (NZ) 534909

(51) Int. Cl. G01N 33/26

(52) U.S. Cl. 436/60

(57) ABSTRACT

A method of assaying the antioxidant activity of a range of plant, animal or synthetic oils and other similar substances in a liquid phase. The method consists in forming an oil-in-water emulsion with a surfactant, adding an ethene generator substrate and an oxidative peroxyl radical solution and measuring the ethene produced by a flow tube technique or atmospheric chemical ionisation methodology.

### CROSS-REFERENCE TO RELATED APPLICATION

This application is a National Stage application of International Application No. PCT/NZ2005/000221, filed on Aug. 22, 2005, which claims priority of New Zealand application No. 534909 filed on Aug. 26, 2004.

### BACKGROUND OF THE INVENTION

In the following description and claims, the term ‘oils’ is intended to include all plant, animal or synthetic oils and other similar substances in a liquid phase including for instance, lipids.

Natural or synthetic dietary or applied oils capable of reducing or eliminating oxidative free radicals in cells and tissues are currently thought to protect against actual or potential oxidative damage in vivo. All the vegetable oils contain different levels of antioxidants (mainly vitamin E, polyphenols and carotenoids) and these contribute to the stability of the oil and to its health-promoting properties.

It is desirable that the oxidative free radical scavenging capacity of oils be directly measured in vitro as distinct from appraising antioxidant activity in purely aqueous based systems. Conventionally the known methods used to monitor the antioxidant activity of oils are of two types. In the first method, a test oil is treated with solvents such as acetone to extract variable amounts of soluble substances in the oil that are then tested by conventional means for their capacity to retard oxygen consumption This method does not provide a measure of the antioxidant capacity of oil because only those components soluble in the solvent are appraised for their antioxidant activity.

The second method heats or otherwise degrades a test oil and after a period of time, measures the by-products of oxidation of that oil (Bertram et al. Polyhydroxy flavonoid antioxidants for edible oils. Structural criteria for activity. Food Chemistry 1983; 10; 47-55). This method also does not provide a direct measure of the antioxidant capacity of the oil. It provides instead, a measure of the conditions required to oxidise some components of the oil in a given time.

The measurement of free radical generation and oxidation, as well as oxidative radical scavenging by naturally occurring or extraneous molecules, is currently both complex and time consuming. In vitro assays of oxidative free radical activity have used three general techniques. The first is based on the time required to obtain maximum oxygen consumption in a system containing the peroxyl radical generator and antioxidants (Wayner et al. “Quantitative measurement of the total, peroxyl radical-trapping antioxidant capability of human blood plasma by controlled peroxidation”. FEBS Lett. 1985; 187; 33-37). The second utilizes fluorescence emission from phycoerythrin which is activated by the radical (Glazer A N. “Fluorescence-based assay for reactive oxygen species: A protective role for carnitine”. FASEB J. 1988; 2: 2487-91). The third technique utilizes the peroxyl radical oxidation of α-keto-γ-methiolbutyric acid (KMBA) to ethene that is then detected and monitored by gas chromatography (Winston G W, et al. A rapid gas chromatographic assay for determining oxyradical scavenging capacity of antioxidants and biological fluids. Free Radical Biology & Medicine 1998; 24: 480-93). In this last method based on the amount of ethene in the headspace, the Total Oxyradical Scavenging Capacities (TOSC) of the antioxidants are determined. A modification of this technique using SIFT-MS in place of gas chromatography with SIFT-MS has been described in WO2004/005911 (SYFT Technologies Ltd).

An end product of radical production is ethene, the detection of which is made possible by the application of SIFT-MS technology. Modifications to the established SIFT technique to convert it to the SIFT-MS technique for analyzing trace components of gas mixtures have been described by P Spanel, D. Smith. Selected Ion Flow tube: a technique for quantitative trace gas analysis of air and breath. Medical and Biol. Comput. 1996, 34, 409-419.

### SUMMARY OF THE INVENTION

It is object of the present invention is to provide a method to enable the rapid evaluation of the antioxidant activity of oils and oil soluble samples.

It is a further object of the invention to evaluate the stability of oils.

It is a further object of the invention to monitor the oxidation and peroxidation rates of oils.

In one form the invention may be said to comprise a method of assaying the antioxidant activity of oils as herein defined, comprising the steps of:

preparing an oil in water emulsion by mixing the oil with a surfactant,

adding an ethene generator substrate to the emulsion,

adding an oxidative peroxyl solution,

measuring the ethene produced by introducing an amount of the ethene produced into a stream of an inert gas containing precursor ions,

ionising the reactive species to form product ions of the reactive species,

analysing the ratio of product ions to precursor ions to provide an absolute concentration of the sample,

analysing each trace gas constituent, and

measuring the amount of ethene produced by flow tube, drift tube or atmospheric chemical ionization methodologies.

Preferably the amount of ethene produced is measured by Selective Ion Flow Tube Mass Spectrometry.

Preferably the surfactant is a non-ionic, block co-polymer surfactant.

Preferably the oil-in-water emulsion is formed by mixing an oxidative peroxyl radical and an ethene generator with the test oil in the presence of the surfactant.

Preferably the peroxyl radicals are generated by the thermal hydrolysis of AAPH (2,2′-Azobis(2-amidinopropane)dihydrochloride) at temperatures between 35° C.-39° C.

Preferably the radical scavenging ability of the oils is determined from their Total Oxyradical Scavenging Capacity (TOSC) values which are measured for different oil samples and are obtained from growth curves of ethene production with time.

### BRIEF DESCRIPTION OF THE DRAWINGS

FIG. 1 shows the rate of reaction of KMBA oxidation by peroxyl radicals in the presence of increasing concentrations of an award winning-good olive oil 1.

FIG. 2 shows the rate of reaction of KMBA oxidation by peroxyl radicals in the presence of increasing concentrations of olive oil 2.

FIG. 3 shows the rate of reaction of KMBA oxidation by peroxyl radicals in the presence of increasing concentrations of canola oil.

FIG. 4 shows the regression of TOSC values of an award winning-good olive oil 1 at different concentrations.

FIG. 5 shows the regression of TOSC values of olive oil 2 at different concentrations.

FIG. 6 shows the regression of TOSC values of canola oil at different concentrations.

### DETAILED DESCRIPTION OF THE PREFERRED EMBODIMENTS

The method of the present invention is based on the known partial inhibition of ethene formation in the presence of antioxidants that compete with KMBA for peroxyl radicals.

The method of assaying the antioxidant activity of oils consists in firstly using an oil-in-water emulsion preferably formed by mixing the oil with the non-ionic block co-polymer surfactant solution. The ethene generator substrate is then added to the emulsion and finally the oxidative peroxyl radical solution is added. Ethene produced by this emulsion mixture is preferably measured by Selective Ion Flow Tube Mass Spectrometry (SIFT-MS).

In addition to measuring ethene by SIFT-MS it is to be understood that the measurements can also be made using other flow tube or drift tube methodologies or by atmospheric chemical ionization methods.

In one aspect, the oil in water emulsion may be prepared by adding 1 to 10% of warm (50-55° C.) oil to 1 to 3% of warm (40-45° C.) P104 non-ionic, block copolymer (polyoxyethylene:polyoxypropylene) surfactant in the phosphate buffer. The mixture is mixed together for 5 to 15 minutes to form a stable emulsion using a rotostator at 40-45° C. The particle size of the emulsion may be analysed using a microtrac X100™ particle analyser. In an experiment it was found that the emulsion contains 93% of the particles that are less than 5 micron in size, 99% of the particles that are less than 10 microns. All the particles were less than 13.1 microns.

Using SIFT-MS and similar flow tube and drift tube methodologies, a small amount of sample gas is introduced into a stream of helium in the flow tube or drift tube containing H₃O⁺, NO⁺ or O₂⁺ precursor ions. The reactive species in the sample are ionised by chemical ionization to form product ions of the reactive species. The ratio of the product ions to precursor ions gives the absolute concentration of the sample. The analysis of each trace gas constituent is completed and displayed very promptly. Using the technology of the present invention it is not necessary to prepare complex samples such as occurs in gas or liquid chromatography and conventional mass spectrometry.

The mixture of surfactant, an oxidative peroxyl radical generator and the oil will produce a reaction cocktail in which the oil and water-based ingredients react to form peroxyl radicals. These radicals generate ethene. If antioxidants are present in the oil, some of the radicals are scavenged by the antioxidants and less ethene is produced. SIFT-MS is utilised to measure the amount of ethene and thus provides a rapid, uncomplicated, inexpensive and accurate measurement of oil antioxidant activity.

SIFT-MS and other flow tube and drift tube technologies utilize chemical ionization, using precursor ions generated by electron impact, by microwave discharge or by glow discharge. The precursor ions may be mass selected using a quadrupole mass filter or else formed in a discharge where they enter a stream of carrier gas which may be helium or some other non-reactant gas. Positive or negative precursor ions may be chosen. The precursor ion must be un-reactive with the bulk gas within which the trace species is carried, but should react rapidly with the trace species of interest. In the present assay, O₂⁺ ions are used to measure ethene. The headspace sample above the oil emulsion is introduced into the carrier gas stream at a calibrated rate via a heated capillary inlet. Alternatively, a mass flow controller or calibrated leak valve may be used. Following their admittance into the carrier gas, the trace components within the sample gas mixture undergo reaction with the precursor ions in the helium bath gas. The concentration of a (or each) trace species, sometimes called a volatile organic compound (VOCs) in the gas mixture, is then calculated from the observed number densities of the precursor and product ions as measured by a second mass analyzer (quadrupole, ion trap or time-of-flight mass spectrometer) in conjunction with a particle multiplier and specialized software interface. In order to calculate the actual partial pressure of the trace species it is essential to know the rate of and products formed by the reaction of the precursor ion with the trace neutral under the conditions within the flow tube.

In a highly preferred form, peroxyl radicals are generated by the thermal hydrolysis of AAPH (2,2′-Azobis(2-amidinopropane)dihydrochloride) at temperatures between 35° C.-39° C. The reaction may be carried out using 0.2 mM of substrate KMBA and 20 mM of AAPH in 100 mM of phosphate buffer at pH 7.4 with the addition of oil in water emulsion. The oil analysis can be carried out using big (1000 ml) bottles or series of small (125 ml) bottles and the measurements for ethene are made at fixed time intervals. The technique may be extended to other radicals such as hydroxyl, and alkoxy and other reactive oxygen species such as hypochlorous acid (HOCl) and peroxynitrite (ONOO⁻) that can oxidise KMBA to ethene.

The analysis by using flow tube or drift tube techniques of ethene generated by the peroxy radical reactivity with KMBA in the emulsion is performed in real time, with no calibration and without standards. In one preferred embodiment of the invention the assay provides an in-vitro method for the sensitive, rapid and continuous, real time, absolute concentration determination and quantification of antioxidant activity of oils and lipid-based products, extracts and biological fluids.

The flow tube and drift tube techniques may also be used for ex-vivo measurement of antioxidant activity in biological samples.

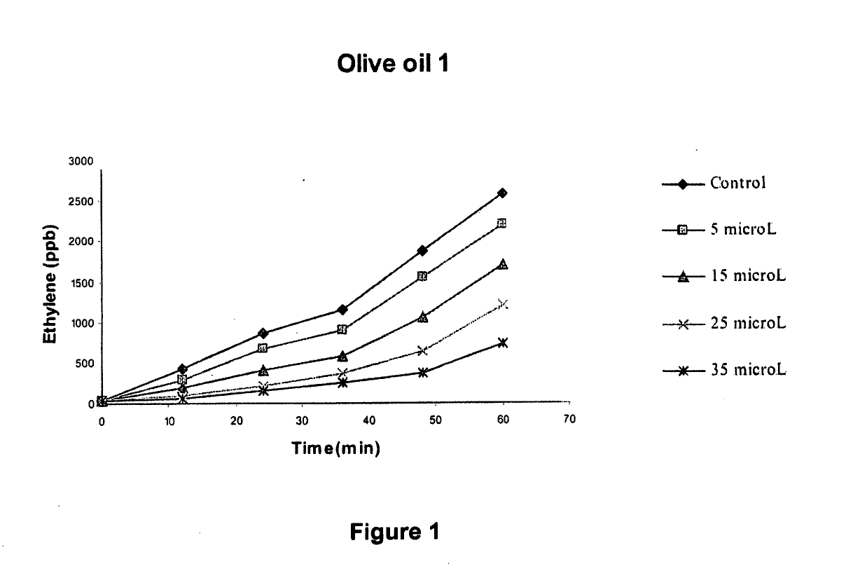

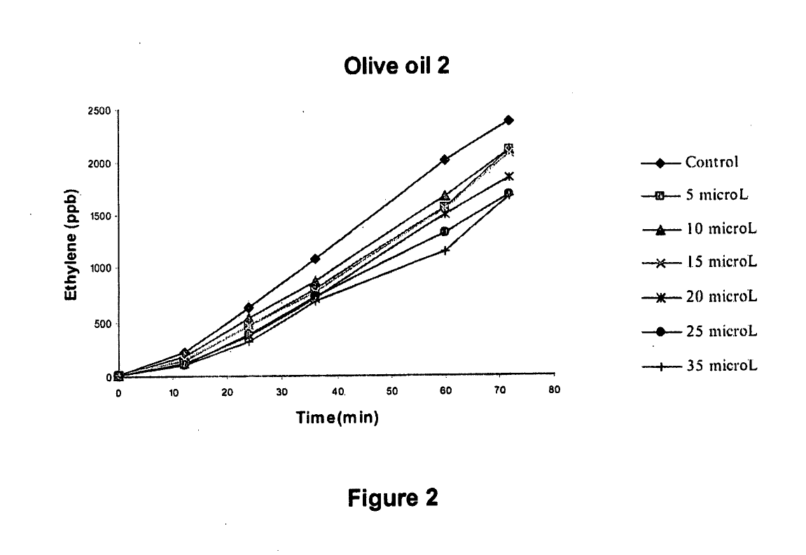

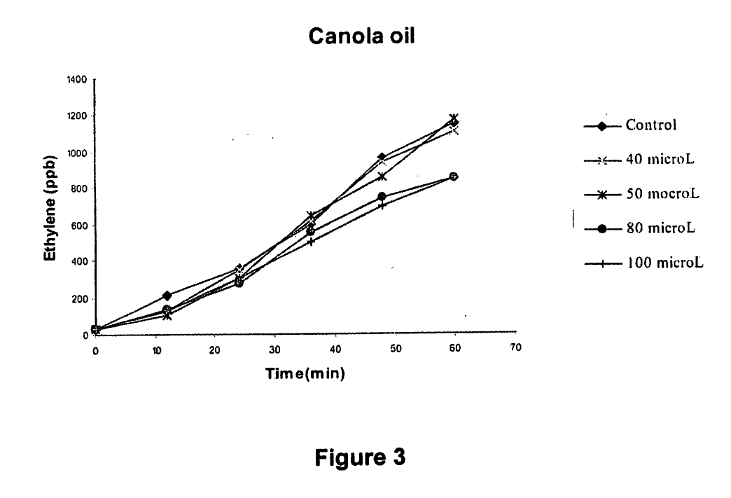

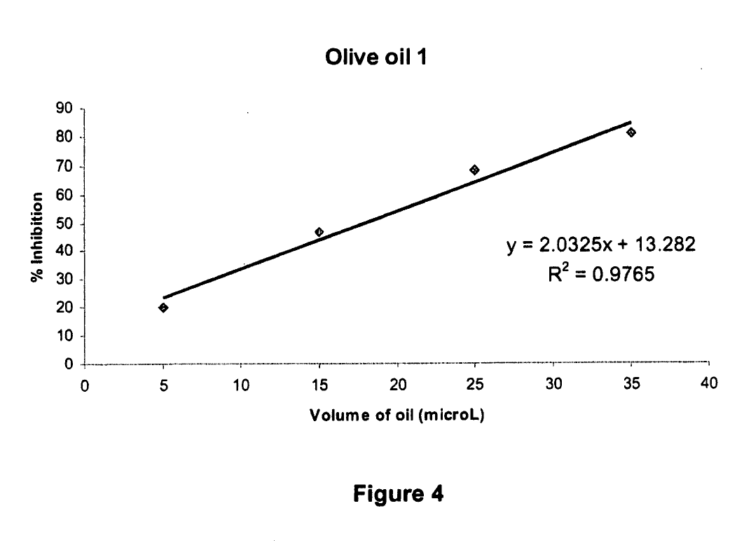

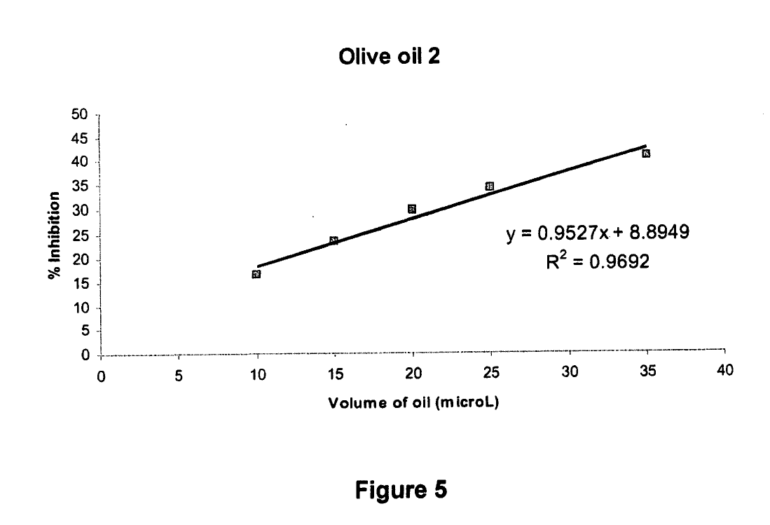

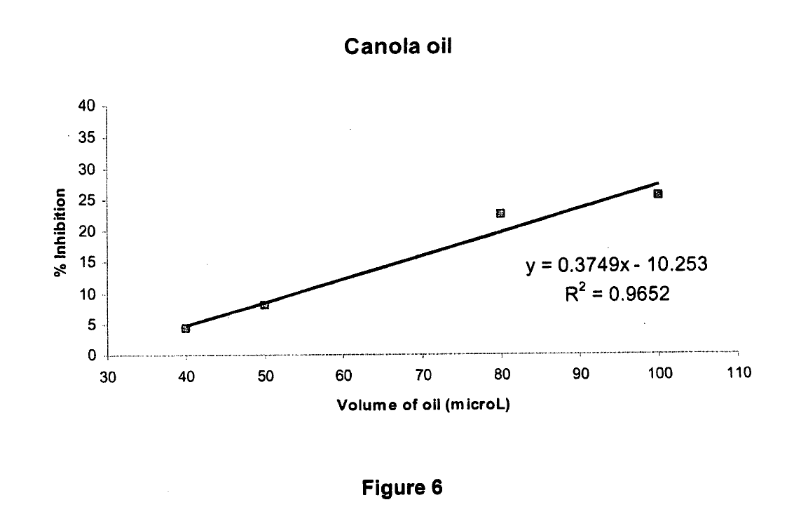

A measure of the radical scavenging ability of the various oils can be found from their Total Oxyradical Scavenging Capacity (TOSC) values. These TOSC values are measured for different oil samples and are obtained from growth curves of ethene production with time. Examples of these curves are shown in FIGS. 1 through 3 for each of the stated oil samples at varying concentrations. The area under each of these growth curves is found for each concentration of the specified antioxidant. The ratio of this area, ∫SA, to the area under the control curve, ∫CA, provides the TOSC value according to
TOSC=100−(∫SA/∫CA)×100
The linear TOSC relationships with concentration derived by this method for each of the oil samples are shown in FIGS. 4 through 6.

In the present invention the reaction rate between O₂⁺ and ethene that is measured may be as follows (Wilson, P. F.; Freeman, C. G.; McEwan, M. J. Reactions of Small hydrocarbons with H₃O⁺, O₂⁺ and NO⁺ Ions. Int. J. Mass Spectrom. 229: 143-149; 2003).
O₂⁺+C₂H₄→C₂H₄⁺+O₂ k=1.0×10⁻⁹ cm³ molecule⁻¹ s⁻¹

The precursor and product ions are scanned over predetermined ranges of mass-to-charge ratio, m/z, for a given time. For this test, the downstream analytical mass filter was switched between the m/z value of the precursor ion (m/z 32) and the m/z value of C₂H₄⁺ (m/z 28) ethene. The partial pressure of ethene in the sample is then calculated immediately, on line, from the precursor and product ion count rates. In this way, rapid changes in ethene concentrations are monitored by SIFT-MS in sequentially assayed headspaces. Total ethene production is measured at the end of the oxidative reaction.

The invention provides an emulsified reaction mixture of aqueous-based oxidative radical generation and substrate catabolism and a lipid-based antioxidant with the end product, ethene, directly measured by using flow tube or drift tube techniques.

The methodology may be automated to determine and measure oxidative radical activity and antioxidant activity in any natural and/or synthetic substance employing the invention.

The method of the present invention provides the following advantages:

a. Fast sensitive method

b. Direct headspace analysis

c. The system is amenable to automation

d. Measures absolute concentrations

e. Ideal for oils, lipids and biological samples

f. Choice of free radical generators (e.g. hydroxyl radical)

g. Choice of other oxidants (e.g. peroxynitrite and HOCl)

h. Measure total antioxidant activity of oil

i. Ideal for oil-based finished products.

The methodology as herein described can be used in a large variety of situations to evaluate the antioxidant potential of oils and oil soluble examples. It may also be used to monitor the oxidation and peroxidation rates of oils and lipids.

A further application of the methodology is to measure the stability of an oil or a lipid sample. Yet another application is the ability to determine the respective qualities of oils and thereby differentiate between a ‘good’ or a ‘bad’ oil, such as for instance olive oil.

Having described preferred embodiments of the invention it will be apparent to those skilled in the art that various changes and alterations can be made to the embodiments and yet still come within the general concept of the invention. All such changes and alterations are intended to be included in the scope of this specification.

1. A method of assaying the antioxidant activity of oils as herein defined, comprising the steps of
preparing an oil in water emulsion by mixing the oil with a surfactant,
adding an ethene generator substrate to the emulsion,
adding an oxidative radical peroxyl solution,
measuring the ethene produced by introducing an amount of the ethene produced into a stream of an inert gas containing precursor ions,
ionising the reactive species to form product ions of the reactive species,
analysing the ratio of product ions to precursor ions to provide an absolute concentration of the sample,
analysing each trace gas constituent, and
measuring the amount of ethene produced by flow tube, drift tube or atmospheric chemical ionization methodologies.

2. The method of claim 1, wherein the amount of ethene produced is measured by Selective Ion Flow Tube Mass Spectrometry.

3. The method as claimed in claim 1 wherein the surfactant is a non-ionic, block copolymer surfactant.

4. The method as claimed in claim 1 wherein the oil-in-water emulsion is formed by mixing said oxidative peroxyl radical solution and said ethene generator with the test oil in the presence of the surfactant.

5. The method as claimed in claim 1 wherein the peroxyl radicals are generated by the thermal hydrolysis of AAPH (2,2′-Azobis(2-amidinopropane)dihydrochloride) at temperatures between 35° C.-39° C.

6. The method as claimed in claim 1 wherein the radical scavenging ability of the oils is determined from their Total Oxyradical Scavenging Capacity (TOSC) values which are measured for different oil samples and are obtained from growth curves of ethene production with time.

7. The method as claimed in claim 1 for evaluating the antioxidant potential of oils and oil soluble examples.

8. The method as claimed in claim 1 for monitoring the oxidation and peroxidation rates of oils and lipids

9. The method as claimed in claim 1 for measuring the stability of an oil or a lipid sample

10. The method as claimed in claim 1 for determining the respective qualities of oils.

11. The method as claimed in claim 10 wherein the oil is an olive oil.
